(19) United States
(12) Patent Application Publication
(10) Pub. No.: US 2008/0160091 A1
(43) Pub. Date: Jul. 3, 2008

(54) COMPOSITE MATERIALS COMPRISED OF CALCIUM COMPOUNDS AND PROTEIN COMPONENTS

(75) Inventors: Christian KROPF, Dusseldorf (DE); Marcel ROTH, Dusseldorf (DE); Ulrike BRUENINGHAUS, Monheim (DE); Stefan MUELLNER, Langenfeld (DE); Albrecht WEISS, Langenfeld (DE); Ulrich SCHOERKEN, Duesseldorf (DE); Lothar KINTRUP, Dusseldorf (DE); Burkhard ESCHEN, Dusseldorf (DE); Amerigo PASTURA, Witten (DE); Peter WUELKNITZ, Leichlingen (DE); Ruediger KNIEP, Langenfeld (DE); Hans LASKA, Langenfeld (DE); Michael MEINDERS, Krefeld (DE); Hans DOLHAINE, Glehn (DE)

Correspondence Address: PAUL & PAUL, 2000 MARKET STREET, PHILADELPHIA, PA, 19103-3229, US

(73) Assignee: HENKEL KOMMANDITGESELLSCHAFT AUF AKTIEN, Dusseldorf (DE)

(21) Appl. No.: 11/864,252

(22) Filed: Sep. 28, 2007

Related U.S. Application Data
(63) Continuation of application No. 10/030,268, filed on Mar. 19, 2002.

(30) Foreign Application Priority Data
Jul. 2, 1999 (DE) 199 30 335.5

(51) Int. Cl. A61K 9/14; A61P 1/02

(52) U.S. Cl. 424/489

(57) ABSTRACT

An oral and dental hygiene product is comprised of a composite material comprising calcium salts which are slightly soluble in water, in the form of nanoparticulate primary particles having a length of from 5 to 150 nm and a cross section of from 2 to 50 nm and protein components selected from the group consisting of proteins, protein hydrolysates and protein hydrolysate derivatives, and 0.1 to 9% by weight of a cleaning agent based on the total weight of the product.

### CROSS-REFERENCE TO RELATED APPLICATIONS

This application is a continuation of U.S. patent application Ser. No. 10/030,268, which was filed on Mar. 19, 2002. Application Ser. No. 10/030,268 was a U.S. National Stage application filed under 35 U.S.C. § 371 of International Application No. PCT/EP00/05813, filed in the European Patent Office on Jun. 23, 2000. This application also claims priority under 35 U.S.C. § 119 of German Application No. DE 199 30 335.5, filed on Jul. 2, 1999.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

This invention relates to composite materials of nanoparticulate, poorly water-soluble calcium salts and protein components of which the composition and fine structure make them particularly suitable for promoting the restoration of bones and dental enamel.

Phosphate salts of calcium have long been added to the formulations of tooth cleaning and dental care preparations both as abrasive components and for promoting the remineralization of dental enamel. This applies in particular to hydroxylapatite and fluorapatite and to amorphous calcium phosphates and to brushite (dicalcium phosphate dihydrate). Calcium fluoride has also been repeatedly described as a constituent of tooth cleaning preparations and as a component for strengthening dental enamel and for the prophylaxis of caries.

The availability of calcium compounds for the desired remineralization is critically determined by the particle size of these poorly water-soluble components which are dispensed in the dental care preparations. Accordingly, it has been proposed to use these poorly soluble calcium salts in the form of very fine particles.

(2) Description of Related Art, Including Information Disclosed Under 37 C.F.R. §§ 1.97 and 1.98

Dental enamel and the supporting tissue of bones consist predominantly of the mineral hydroxylapatite. In the biological formation process, hydroxylapatite attaches itself in an ordered manner to the protein matrix in the bone or tooth which consists predominantly of collagen. The development of the hard and very strong mineral structures is controlled by the so-called matrix proteins which are formed by other proteins besides collagen. These other proteins attach themselves to the collagen and thus effect a structured mineralization process which is also known as biomineralization.

In the restoration of bone material, an important part is played by so-called bone substitutes which promote the natural biomineralization process. These substitutes are also required for coating implants to establish firm bonds between bone and implant which are even capable of transmitting tensile forces. Of particular significance in this regard are coatings with high bioactivity, which lead to an effective compound osteogenesis. According to the prior art as described, for example, by G. Willmann in Mat.-wiss. u. Werkstofftech. 30 (1999), 317, hydroxylapatite is generally applied to implants. The disadvantage of this approach, besides the often inadequate acceleration of the biomineralization process, lines in the flaking of the hydroxylapatite layers and their unsatisfactory chemical stability.

There are certain applications which require bone substitute materials that are capable of being injected as liquids. A particularly small particle size is required for such applications but, unfortunately, cannot be satisfactorily achieved with conventional bone substitutes.

Among known bone substitutes, composites of hydroxylapatite and collagen are of particular interest because they imitate the composition of natural bone material. A similar situation prevails in the restoration of tooth material, of which about 95% consists of hydroxylapatite.

Composite materials of the described type can be obtained by synthetic methods as described, for example, by B. Flautre et al. in J. Mater. Sci.: Mater. in Medicine 7 (1996), 63. However, the particle size of the calcium salts in these composites is above 1,000 nm, which is too large for a satisfactory biological effect as remineralizing agents.

By contrast, R. Z. Wang et al., J. Mater. Sci. Lett. 14 (1995), 490, describe a process for the production of a composite material of hydroxylapatite and collagen in which hydroxylapatite with a particle size of 2 to 10 nm is deposited in uniformly distributed form onto the collagen matrix. The composite material is said to have better biological activity than other hydroxylapatite/collagen composites known from the prior art by virtue of the particle fineness of the hydroxylapatite. As described in the following, the composite material described by R. Z. Wang et al. also fails to adequately meet the need for composite materials which imitate the composition and microstructure of natural bone and tooth material and which are suitable in every respect for remineralizing these natural materials.

Protein-containing composite materials known from the prior art contain proteins of animal origin, more particularly proteins obtained from bovine material. However, for some years now, there has been an increasing demand, particularly in the cosmetics field, for products which are entirely free from ingredients of animal origin. Accordingly, there is also a need for composite materials which do not contain any protein components of animal origin.

Another disadvantage of protein-containing composite materials known from the prior art lies in their often complicated production. For example, in the production of the hydroxylapatite/collagen composite described by R. Z. Wang et al., insoluble collagen has to be handled and dispersed in very large quantities of solvent which, on a large scale, is very difficult. This process creates additional problems in regard to the disposal of the wastewaters accumulating during the production process.

In addition, the protein-containing composite materials known from the prior art show unfavorable dispersibility through the presence of insoluble and/or high molecular weight protein components and are difficult to incorporate in the formulations required for their commercial application or show unsatisfactory dispersion stability in the preparations used.

### BRIEF SUMMARY OF THE INVENTION

It has now been found that certain composite materials are suitable for overcoming the above-described disadvantages of the prior art.

The present invention relates to composite materials comprising
a) poorly water-soluble calcium salts selected from phosphates, fluorides and fluorophosphates which—if desired—may additionally contain hydroxyl and/or carbonate groups, the calcium salts being present in the form of nanoscale primary particles with a mean particle diameter of 10 to 300 nm, and
b) protein components selected from proteins, protein hydrolyzates and protein hydrolyzate derivatives.

### BRIEF DESCRIPTION OF THE SEVERAL VIEWS OF THE DRAWINGS

FIG. 1 is a TEM micrograph of a composite according to the invention comprising hydroxylapatite and Type A gelatine.

FIG. 2 is a TEM micrograph of the composite of hydroxylapatite and Type A gelatine after the hydroxylapatite has been dissolved out with ethylenediamine tetraacetate.

### DETAILED DESCRIPTION OF THE INVENTION

Composite materials in the context of the invention are understood to be composite materials which comprise the components mentioned in a) and b) and represent microscopically heterogeneous, but macroscopically homogeneous-looking aggregates and in which the primary particles of the calcium salts are associated onto the skeleton of the protein component. The percentage content of the protein components in the composite materials is between 0.1 and 60% by weight and preferably between 0.5 and 10% by weight, based on the total weight of the composite materials.

Primary particles are understood to be the crystallites, i.e., the microcrystals of the calcium salts mentioned. The particle diameter is understood here to be the diameter of the particles in the direction of their greatest length, while the mean particle diameter is understood to be a value averaged over the total quantity of the composite. The particle diameter may be determined by any method known to the expert, for example, by the method of transmission electron microscopy (TEM).

The mean particle diameter of the nanoscale primary particles is in the range from 10 to 150 nm. In a particularly preferred embodiment, the primary particles are present in the form of rodlet-like particles with a thickness of 2 to 50 nm and a length of 10 to 150 nm. Thickness is understood here to be the smallest diameter of the rodlets and length, their largest diameter.

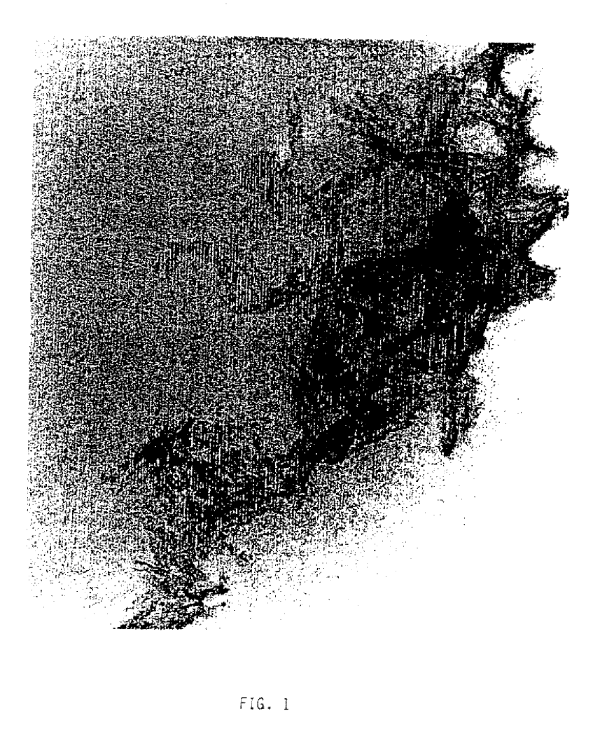

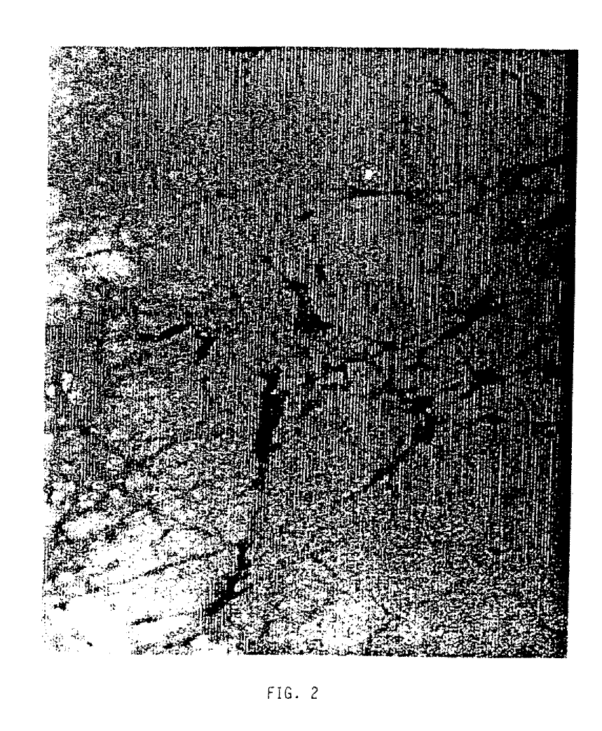

The three-dimensional structure of the composite materials according to the invention of a protein component and the poorly soluble nanoparticulate calcium salts is illustrated by way of example by the TEM micrograph in FIG. 1 of a composite material of hydroxylapatite and type A gelatine (magnification 200,000×; 1 cm in FIG. 1 corresponds to 40 nm). The rodlet-like nanoparticles of hydroxylapatite are superimposed on the high molecular weight protein component, which assumes a three-dimensional structure essentially determined by its amino acid sequence. In other words, the nanoparticles so to speak reproduce the three-dimensional structure of the protein component. This is illustrated in FIG. 2, which is a TEM micrograph of the type A gelatine skeleton of the same composite material after the hydroxylapatite has been dissolved out with a solution of ethylenediamine tetraacetate (magnification 56,000×; 1.1 cm in FIG. 2 corresponds to 200 nm). The way in which the inorganic particles are attached to the basic skeleton of the protein component is determined by the primary structure (amino acid sequence) and—depending on the nature of the protein component—by its secondary, tertiary and quaternary structure. It has surprisingly been found that the spatial distribution and the quantitative extent of the attachment of the inorganic nanoparticles to the protein component can be influenced by the type and quantity of the amino acids present in the protein component, and hence by the choice of the protein components. Thus, a particularly high degree of charging with the poorly soluble calcium salt can be achieved, for example, through the choice of protein components which are rich in the amino acids aspartic acid, glutamic acid or cysteine. In addition, depending on the spatial distribution of these amino acids in the protein skeleton, the charging of the protein component with the poorly soluble calcium salt can be spatially structured in a certain way.

Accordingly, the composite materials according to the invention are structured composite materials, in contrast to the hydroxylapatite/collagen composite described by R. Z. Wang et al., in which uniformly distributed hydroxylapatite nanoparticles are present. Another crucial difference between the subject of the present invention and the prior art lies in the size and morphology of the inorganic component. The hydroxylapatite particles present in the hydroxylapatite/collagen composite described by R. Z. Wang et al. have a size of 2 to 10 nm. Hydroxylapatite particles in this size range can be assigned to the range of amorphous or partly X-ray-amorphous materials.

Surprisingly, it was possible through the present invention to produce composite materials containing crystalline inorganic nanoparticles in which the nanoparticles have a microscopically clearly discernible crystalline morphology. FIG. 1 shows the rodlet-like structure of the inorganic nanoparticles. It has also been found that the structured composite materials according to the invention, in contrast to the prior art, lead to a particularly effective biomineralization process. It is assumed that this is associated with the microstructure of the composite material and, more particularly, with the size and morphology of the calcium salt crystals. Thus, it is assumed that the longitudinal axis of the calcium salt nanoparticles represents a preferential direction for further crystal growth during the biomineralization process.

Poorly water-soluble salts are salts of which less than 1 g/l dissolves at 20°. Preferred calcium salts are calcium hydroxyphosphate (Ca₅[OH(PO₄)₃]) or hydroxylapatite, calcium fluorophosphates (Ca₅[F(PO₄)₃]) or fluoropatite, fluorine-doped hydroxylapatite with the general composition Ca₅(PO₄)₃(OH,F) and calcium fluoride (CaF₂) or fluorite (fluorspar).

One or more salts in admixture selected from the group of phosphates, fluorides and fluorophosphates, which, if desired, may additionally contain hydroxyl and/or carbonate groups, may be present as calcium salt in the composite materials according to the invention.

Any proteins may be used as proteins in accordance with the invention, irrespective of their origin or their production. Examples of proteins of animal origin are keratin, elastin, collagen, fibroin, albumin, casein, whey protein, placenta protein. Of these, collagen, keratin, casein, and whey protein are preferred for the purposes of the invention. Proteins of vegetable origin, such as, for example, wheat or wheat germ protein, rice protein, soya protein, oat protein, pea protein, potato protein, almond protein and yeast protein, may also be preferably used for the purposes of the invention.

Protein hydrolyzates in the context of the present invention are understood to be degradation products of proteins such as, for example, collagen, elastin, casein, keratin, almond, potato, wheat, rice and soya protein, which are obtained by acidic, alkaline and/or enzymatic hydrolysis of the proteins themselves or their degradation products, such as gelatine, for example. Any hydrolytically acting enzymes, such as alkaline proteases, for example, may be used for the enzymatic degradation. Other suitable enzymes and enzymatic hydrolysis processes are described, for example, in K. Drauz and H. Waldmann, Enzyme Catalysis in Organic Synthesis, VCH Verlag, Weinheim 1975. During their degradation, the proteins are split into relatively small subunits, the degradation process proceeding via the stages of the polypeptides through the oligopeptides up to the individual amino acids. In the context of the present invention, low-degradation protein hydrolyzates include, for example, the gelatine preferred for the purposes of the invention, which may have molecular weights in the range from 15,000 to 250,000 D. Gelatine is a polypeptide which is mainly obtained by hydrolysis of collagen under acidic conditions (type A gelatine) or alkaline conditions (type B gelatine). The gel strength of the gelatine is proportional to its molecular weight, i.e., a gelatine hydrolyzed to a relatively high degree gives a solution of relatively low viscosity. The gel strength of the gelatine is expressed in Bloom values. In the enzymatic hydrolysis of the gelatine, the polymer size is greatly reduced, which leads to very low Bloom values.

According to the invention, other preferred protein hydrolyzates are the protein hydrolyzates used in the cosmetics field with an average molecular weight of 600 to 4,000 and preferably 2,000 to 3,500. Overviews of the production and use of protein hydrolyzates have been published, for example, by G. Schuster and A. Domsch in Seifen, Ole, Fette, Wachse, 108, (1982) 177 and Cosm. Toil. 99, (1984) 63, by H. W. Steisslinger in Parf. Kosm. 72, (1991) 556 and by F. Aurich et al. in Tens. Surf. Det. 29, (1992) 389. According to the invention, protein hydrolyzates of collagen, keratin, casein and vegetable proteins, for example, those based on wheat gluten or rice protein, of which the production is described in German patents DE 19502167 C1 and DE 19502168 C1 (Henkel), are preferably used.

Protein hydrolyzate derivatives in the context of the present invention are understood to be chemically and/or chemoenzymatically modified protein hydrolyzates such as, for example, the compounds known by the INCI names of Sodium Cocoyl Hydrolyzed Wheat Protein, Laurdimonium Hydroxypropyl Hydrolyzed Wheat Protein, Potassium Cocoyl Hydrolyzed Collagen, Potassium Undecylenoyl Hydrolyzed Collagen and Lauridmonium Hydroxypropyl Hydrolyzed Collagen. According to the invention, derivatives of protein hydrolyzates of collagen, keratin and casein and vegetable protein hydrolyzates such as, for example, Sodium Cocoyl Hydrolyzed Wheat Protein or Laurdimonium Hydroxypropyl Hydrolyzed Wheat Protein are preferred.

Other examples of protein hydrolyzates and protein hydrolyzate derivatives which fall within the scope of the present invention are described in CFTA 1997 International Buyers' Guide, John. A. Wenninger et al. (Ed.), The Cosmetic, Toiletry and Fragrance Association, Washington D.C. 1997, 686-688.

In each of the composite materials according to the invention, the protein component may be formed by one or more substances selected from the group of proteins, protein hydrolyzates and protein hydrolyzate derivatives.

Preferred protein components are any structure-forming proteins, protein hydrolyzates and protein hydrolyzate derivatives by which are meant protein components which, through their chemical constitution, form certain three-dimensional structures that are known to the expert from protein chemistry as secondary, tertiary or even quaternary structures.

In another embodiment of the present invention, the nanoscale calcium salt primary particles present in the composite materials may be encapsulated in one or more surface modifiers.

In this way, it is possible, for example, to facilitate the production of composite materials in cases where the nanoparticulate calcium salts are difficult to disperse. The surface modifier is adsorbed onto the surface of the nanoparticles and modifies them to the extent that the dispersibility of the calcium salt increases and the nanoparticles are prevented from agglomerating.

In addition, the structure of the composite materials and the charging of the protein component with the nanoparticulate calcium salt can be influenced by surface modification. In this way, it is possible where the composite materials are used in remineralization processes to influence both the course and the speed of the remineralization process.

Surface modifiers in the context of the present invention are understood to be substances which physically adhere to the surface of the fine particles but do not react chemically with them. The individual molecules of the surface modifiers adsorbed to the surface are substantially free from intermolecular bonds between one another. Surface modifiers are understood, in particular, to be dispersants. Dispersants are also known to the expert by such names as, for example, emulsifiers, protective colloids, wetting agents, detergents, etc.

Suitable surface modifiers are, for example, emulsifiers of the nonionic surfactant type from at least one of the following groups:
products of the addition of 2 to 30 moles ethylene oxide and/or 0 to 5 moles propylene oxide onto linear fatty alcohols containing 8 to 22 carbon atoms, onto fatty acids containing 12 to 22 carbon atoms and onto alkylphenols containing 8 to 15 carbon atoms in the alkyl group;
C_{12/18} fatty acid monoesters and diesters of addition products of 1 to 30 moles of ethylene oxide with glycerol;
glycerol mono- and diesters and sorbitan mono- and diesters of saturated and unsaturated fatty acids containing 6 to 22 carbon atoms and ethylene oxide addition products thereof;
alkyl mono- and oligoglycosides containing 8 to 22 carbon atoms in the alkyl group and ethoxylated analogs thereof;
addition products of 15 to 60 moles of ethylene oxide with castor oil and/or hydrogenated castor oil;
polyol esters and, in particular, polyglycerol esters such as, for example, polyglycerol polyricinoleate, polyglycerol poly-12-hydroxystearate or polyglycerol dimerate. Mixtures of compounds from several of these classes are also suitable;
addition products of 2 to 15 moles of ethylene oxide with castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example, sorbitol), alkyl glucosides (for example, methyl glucoside, butyl glucoside, lauryl glucoside) and polyglucosides (for example cellulose);
mono-, di and trialkyl phosphates and mono-di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol according to DE-PS 11 65 574 and/or mixed esters of fatty acids containing 6 to 22 carbon atoms, methyl glucose and polyols, preferably glycerol or polyglycerol, and
polyalkylene glycols.

The addition products of ethylene oxide and/or propylene oxide with fatty alcohols, fatty acids, alkyl phenols, glycerol monoesters and diesters and sorbitan monoesters and diesters of fatty acids or with castor oil are known commercially available products. They are homolog mixtures of which the average degree of alkoxylation corresponds to the ratio between the quantities of ethylene oxide and/or propylene oxide and substrate with which the addition reaction is carried out.

C_{8/18} mono- and oligoglycosides, their production and their use are known from the prior-art literature. They are produced, in particular, by reacting glucose or oligosaccharides with primary alcohols containing 8 to 18 carbon atoms. So far as the glycoside component is concerned, both monoglycosides where a cyclic sugar unit is attached to the fatty alcohol by a glycoside bond and oligomeric glycosides with a degree of oligomerization of preferably up to about 8 are suitable. The degree of oligomerization is a statistical mean value on which a homolog distribution typical of such technical products is based.

Typical examples of anionic emulsifiers are soaps, alkyl benzenesulfonates, alkanesulfonates, olefin sulfonates, alkylether sulfonates, glycerol ether sulfonates, α-methyl ester sulfonates, sulfofatty acids, alkyl sulfates, alkyl ether sulfates such as, for example, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl glutames and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazoliens containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. According to the invention, other suitable emulsifiers besides ampholytic surfactants are quaternary emulsifiers, those of the esterquat type, preferably methyl-quaternized difatty acid triethanolamine ester salts, being particularly preferred.

Protective colloids suitable as surface modifiers are, for example, natural water-soluble polymers such as, for example, gum arabic, starch, water-soluble derivatives of water-insoluble, polymeric natural materials such as, for example, cellulose ethers, such as methyl cellulose, hydroxyethyl cellulose, carboxymethyl cellulose or modified carboxymethyl cellulose, hydroxyethyl starch or hydroxypropyl guar, and synthetic water-soluble polymers such as, for example, polyvinyl pyrrolidone, polyalkylene glycols, polyaspartic acid and polyacrylates.

The surface modifiers are used in a concentration of generally 0.1 to 50% by weight and preferably 1 to 20% by weight, based on the calcium salts.

Preferred surface modifiers are, above all, the nonionic surfactants in a quantity of 1 to 20% by weight, based on the weight of the calcium salt. Nonionic surfactants of the alkyl-C₈₋₁₆-(oligo)glucoside type and hydrogenated castor oil ethoxylate type have proved to be particularly effective. The composite materials according to the invention are prepared by precipitation reactions from aqueous solutions of water-soluble calcium salts and aqueous solutions of water-soluble phosphate and/or fluoride salts, the precipitation being carried out in the presence of protein components. This is preferably done by adding the protein components in pure, dissolved or colloidal form to the alkaline aqueous phosphate and/or fluoride salt solution or to the alkaline solution of the calcium salt before the precipitation reaction. Alternatively, the protein components may be initially introduced in pure, dissolved or colloidal form followed by addition of the alkaline calcium salt solution and the alkaline phosphate and/or fluoride salt solution either successively in any order or at the same time.

In the production process according to the invention, the individual components may be fitted together in any order. Ammonia is preferably used as the alkalizing agent.

In another variant of the production process according to the invention, the precipitation is carried out from an acidic solution of a water-soluble calcium salt together with a stoichiometic quantity of a water-soluble phosphate and/or fluoride salt or from an acidic solution of hydroxylapatite with a pH below 5, preferably at a pH below 3, by raising the pH with aqueous alkali or ammonia in the presence of the protein components.

In another variant of the process, the protein components are added, preferably in dissolved or dispersed form, to nanoparticulate calcium salts in pure or dispersed form or to dispersions of nanoparticulate calcium salts prepared by precipitation reactions from aqueous solutions of water-soluble calcium salts and aqueous solutions of water-soluble phosphate and/or fluoride salts, the addition being made in any order.

The solution or dispersion of the protein component is preferably introduced first and a dispersion of the nanoparticulate calcium salt subsequently added.

In all processes involving the precipitation of apatite, it is advisable to keep the pH below 5 and preferably below 3. In all the production processes mentioned, the dispersion of the composite material formed may, if desired, be separated off from the solvent and the other constituents of the reaction mixture by methods known to the expert, such as filtration or centrifugation, for example, and isolated in solvent-free form by subsequent drying, for example, by freeze-drying.

In all the production processes, water is preferably used as the solvent, although organic solvents, for example C₁₋₄ alcohols or glycerol, may also be used in individual steps of the production process.

The production of the composite materials according to the invention in which the primary particles of the calcium salts are surface-modified may be carried out by precipitation processes similar to those described above, except that the precipitation of the nanoparticulate calcium salts or the composite materials is carried out in the presence of one or more surface modifiers.

In a preferred embodiment, the surface-modified nanoparticulate calcium salts are first produced by a precipitation reaction between aqueous solutions of calcium salts and aqueous solutions of phosphate and/or fluoride salts in the presence of the surface modifiers. The surface-modified nanoparticulate calcium salts may then be freed from accompanying products of the reaction mixture, for example, by concentration under reduced pressure and subsequent dialysis. A dispersion of the surface-modified calcium salt with any desired solids content may additionally be prepared by removing the solvent. The composite material of surface-modified calcium salt and protein components is then formed by addition of the protein components in pure, dissolved or colloidal form—again in any order—and, if necessary, after-reaction for 1 to 100 minutes at elevated temperature, preferably in the range from 50 to 100° C.

Other processes such as those described in German Patent Application DE 19858662.0 may be used to produce dispersions of surface-modified calcium salts.

The composite materials according to the invention, more particularly, those of hydroxylapatite, fluorapatite and calcium fluoride, are suitable as a remineralizing component for the production of tooth cleaning and/or dental care compositions. The structured form of the composites and the particle size of the calcium compounds present in them enable the effect of strengthening dental enamel and sealing lesions and dentine channels to be developed particularly quickly and completely. The tooth cleaning and dental care compositions may be formulated, for example, as pastes, liquid creams, gels or mouthwashes. The composite materials according to the invention are readily dispersed, even in liquid preparations, and remain stably dispersed, i.e., have no tendency to sediment.

A preferred embodiment are toothpastes containing silica, polishes, humectants, binders and flavors which contain 0.1 to 10% by weight of composite materials according to the invention containing nanoparticulate calcium salts from the group consisting of hydroxylapatite, fluorapatite and calcium fluoride.

The tooth cleaning and dental care preparations may contain the usual components and auxiliaries of such compositions in the usual quantities. For toothpastes, these are, for example:
abrasives and polishes such as, for example, chalk, silicas, aluminium hydroxide, aluminium silicates, calcium pyrophosphate, dicalcium phosphate, insoluble sodium metaphosphate or synthetic resin powder;
humectants such as, for example, glycerol, 1,2-propypene glycol, sorbitol, xylitol and polyethylene glycols;
binders and consistency factors, for example, natural and synthetic water-soluble polymers and water-soluble derivatives of natural materials, for example, cellulose ethers, layer silicates, fine-particle silicas (aerogel silicas, pyrogenic silicas);
flavors, for example, peppermint oil, spearmint oil, eucalyptus oil, aniseed oil, fennel oil, caraway seed oil, menthyl acetate, cinnamaldehyde, anethol, vanillin, thymol and mixtures of these and other natural and synthetic flavors;
sweeteners such as, for example, saccharin sodium, sodium cyclamate, aspartame, acesulfan K, stevioside, monellin, glycyrrhicin, dulcitol, lactose, maltose or fructose;
preservatives and antimicrobial agents such as, for example, p-hydroxybenzoic acid esters, sodium sorbate, triclosan, hexachlorophene, phenyl salicylic acid ester, thymol, etc.;
pigments such as, for example, titanium dioxide or pigment dyes for producing colored stripes;
buffers such as, for example, primary, secondary or tertiary alkali metal phosphates or citric acid/sodium citrate; and
wound-healing and anti-inflammatory agents such as, for example, allantoin, urea, azulene, panthenol, acetyl salicylic acid derivatives, plant extracts, vitamins, for example, retinol or tocopherol.

The composite materials according to the invention, more particularly, those of hydroxylapatite and fluorapatite, are capable of inducing or promoting biomineralization in bone tissue. Accordingly, they are also suitable as a biomineralizing component for the production of compositions for restoring or reforming bone material, for example, compositions for the treatment of bone defects and bone fractures and for promoting the “growing in” of implants.

For coating implants, the composite materials according to the invention may be applied, for example, by the standard methods known to the expert of dip coating or plasma spraying.

For use as injectable bone substitute materials, the composite materials according to the invention may be combined with suitable other substances such as, for example, glycosaminoglycans or proteins, and with suitable solvents and auxiliaries such as, for example, a dilute aqueous phosphate buffer.

The following Examples are intended to illustrate the invention.

### EXAMPLES

### 1. Preparation of Protein Solutions or Dispersions

1.1 Type A Gelatine:
100 ml water were added to 10 g type A gelatine (gelatine obtained by acidic hydrolysis of pig skin) and boiled once in a microwave.

1.2 Type A Gelatine and Casein:
100 ml water and 10 ml of the supernatant phase of a casein solution saturated at 20° C. and then centrifuged at 5,000 r.p.m. were added to 10 g type A gelatine and boiled once in a microwave.

1.3 Hydrolyzate of Type A Gelatine:
100 ml water and the alkaline protease Savinase (Manufacturer: Novo Nordisk) in a concentration of 0.005% enzyme dry matter, based on the gelatine dry matter, were added to 10 g type A gelatine. After stirring for 20 h at 20° C., the whole was boiled once in a microwave.

1.4 Hydrolyzate of Type A Gelatine and Casein:
100 ml water were added to 10 g type A gelatine and 1 g casein and the whole was hydrolyzed overnight at room temperature with the alkaline protease Savinase (Manufacturer: Novo Nordisk) in a concentration of 0.005% enzyme dry matter, based on the dry matter of the protein components, boiled once in a microwave and then filtered.

1.5 Type B Gelatine:
100 ml water were added to 10 g type B gelatine (gelatine obtained by alkaline hydrolysis of cowhide) and boiled once in a microwave.

1.6 Type B Gelatine and Casein:
100 ml water and 10 ml of the supernatant phase of a casein solution saturated at 20° C. and then centrifuged at 5,000 r.p.m. were added to 10 g type B gelatine and boiled once in a microwave.

1.7 Hydrolyzate of Type B Gelatine:
100 ml water and the alkaline protease Savinase (Manufacturer: Novo Nordisk) in a concentration of 0.005% enzyme dry matter, based on the gelatine dry matter, were added to 10 g type B gelatine. After stirring for 20 h at 20° C., the whole was boiled once in a microwave.

1.8 Hydrolyzate of Type B Gelatine and Casein:
100 ml water were added to 10 g type B gelatine and 1 g casein and the whole was hydrolyzed overnight at room temperature with the alkaline protease Savinase (Manufacturer: Novo Nordisk) in a concentration of 0.005% enzyme dry matter, based on the dry matter of the protein components, boiled once in a microwave and then filtered.

### 2. Production of Composite Materials by Precipitation Reactions in the Presence of the Protein Components

2.1 Composite Material of Hydroxylapatite and Type A Gelatine:
2.21 g calcium chloride were dissolved in 137 ml deionized water, heated to 25° C. and adjusted to pH 11 with 25% by weight aqueous ammonia solution. 20 ml of the protein solution prepared in accordance with Example 1.1 and heated in a water bath to 30-40° C. were then added with vigorous stirring. An aqueous solution of 1.58 g diammonium hydrogen phosphate in 26 ml deionized water, which had been heated to 25° C. and adjusted to pH 11 with ammonia solution, was then slowly added dropwise over a period of 1 hour during which the composite material was precipitated. At the beginning of the dropwise addition, the pH was 10.4 and was kept at about 10 by the addition of more ammonia solution. After a reaction time of 20 h (25° C., with stirring), the pH of the aqueous suspension had fallen to 9.5. The composite material precipitated was removed by centrifuging at 5,000 r.p.m., washed with deionized water heated to ca. 30-40° C. and freeze-dried. 2.2 g composite material were obtained. Elemental analysis revealed a carbon content of 2.3%, which corresponds to a content of protein material of 5.6̂ by weight, based on the total quantity of composite material.

2.2-2.8 Composite Materials of Hydroxylapatite and Other Protein Components.
Composite materials of hydroxylapatite and the other protein components described in 1.2 to 1.8 were obtained in the same way as described in Example 2.1.

### 3. Production of Composite Materials by Incorporating Dispersions of Surface-Modified Calcium Salts Ion Protein Components

3.1 Composite Material of Hydroxylapatite and Gelatine Bloom 300:
Solutions A and B were first separately prepared.

Solution A:
25.4 g calcium nitrate tetrahydrate and 8.50 g diammonium hydrogen phosphate were separately dissolved in 100 g deionized water. The two solutions were then combined to form a white precipitate. After addition of 10 ml of 37% by weight of HCl, a clear solution was obtained.

Solution B:
200 ml deionized water, 200 ml 25% by weight aqueous ammonia solution and 20 g Plantacare® 1200 were combined and cooled to 0° C. in an ice bath. Solution A was added to solution B with vigorous stirring, a hydroxylapatite precipitate being formed. After excess ammonia had been removed, the dispersion was purified by dialysis. The dispersion was then concentrated by evaporation in a rotary evaporator by determining the quantity of water separated to such an extent that the dispersion had a solids content, expressed as hydroxylapatite, of 7.5% by weight.
This dispersion was added at room temperature to 100 ml of a 10% by weight aqueous solution of Gelatine Bloom 300 (Manufacturer: Fluka) prepared in accordance with Example 1.1, then heated to 80° C. and stirred at that temperature for 5 minutes. The whole was then left to solidify at room temperature to form the composite material.

### 4. Tooth Creams Containing Calcium Salt Composite Materials

| | Formulation Examples | | | |
|---|---|---|---|---|
| | 4.1 | | 4.2 | |
| Sident ® 8 | 10.0% | by weight | 10.0% | by weight |
| Sident ® 22S | 7.0% | by weight | 7.0% | by weight |
| Sipernat ® 320DS | 0.8% | by weight | 0.8% | by weight |
| Composite material of | 5.0% | by weight | — | |
| Example 2.1 | | | | |
| Composite material of | — | | 5.0% | by weight |
| Example 3.1 | | | | |
| Polywachs ® 1550 | 2.0% | by weight | 2.0% | by weight |
| Texapon ® K1296 | 1.5% | by weight | 1.5% | by weight |
| Titanium dioxide | 1.0% | by weight | 1.0% | by weight |
| Cekol ® 500 T | 1.0% | by weight | 1.0% | by weight |
| Na fluoride | 0.33% | by weight | 0.33% | by weight |
| Na benzoate | 0.25% | by weight | 0.25% | by weight |
| Flavor | 1.0% | by weight | 1.0% | by weight |
| Tagat ® S | 0.2% | by weight | — | |
| Na saccharinate | 0.15% | by weight | 0.15% | by weight |
| Trisodium phosphate | 0.10% | by weight | 0.10% | by weight |
| Sorbitol (70% in water) | 31.0% | by weight | 31.0% | by weight |
| Water | to 100% | by weight | to 100% | by weight |

The following commercial products were used:

### Plantacare® 1200:

C₁₂₋₁₆ alkylglycoside, ca. 50% in water,
Manufacturer: HENKEL KGaA

### Sident® 8:

Synth. amorph. silica, BET 60 m²/g, compacted bulk density: 350 g/l
Manufacturer: DEGUSSA

### Sident® 22S:

Hydrogel silica, BET 140 m²/g, compacted bulk density: 100 g/l
Manufacturer: DEGUSSA

### Polywachs® 1550:

Polyethylene glycol, MW: 1550, softening point: 45-50° C.
Manufacturer: RWE/DEA

### Texapon® K 1296:

Sodium lauryl sulfate powder
Manufacturer: Henkel KGaA

### Cekol® 500 T:

Sodium carboxymethyl cellulose, viscosity (2% in water, Brookfield LVF 20° C.): 350-700 mPa·s
Supplier: Nordmann-Rassmann

### Tagat® S:

Polyoxyethylene-(20)-glyceryl monostearate
Manufacturer: Tego Cosmetics (Goldschmidt)

1. An oral and dental hygiene product comprising
a) a composite material comprising
calcium salts which are slightly soluble in water, in the form of nanoparticulate primary particles having a length of from 5 to 150 nm and a cross section of from 2 to 50 nm and
protein components selected from the group consisting of proteins, protein hydrolysates and protein hydrolysate derivatives, and
b) 0.1 to 9% by weight of a cleaning agent based on the total weight of the product.

2. The oral and dental hygiene product as claimed in claim 1, characterized in that the calcium salts are selected from the group consisting of fluoroapatite, hydroxyapatite and mixtures thereof.

3. The oral and dental hygiene product as claimed in claim 1, characterized in that the protein component is selected from the group consisting of collagen, gelatin, keratin, casein, wheat protein, rice protein, soybean protein, almond protein and their hydrolysates and hydrolysate derivatives.

4. The oral and dental hygiene product as claimed in claim 1, characterized in that the content of the composite material is from 0.01 to 10% by weight based on the total weight of the product.

5. The oral and dental hygiene product as claimed in claim 1, characterized in that the nanoparticulate primary particles are rod-like.

6. The oral and dental hygiene product as claimed in claim 1, characterized in that the nanoparticulate primary particles form rod-like crystals having a length-to-width ratio of 3 to <5.

7. The oral and dental hygiene product as claimed in claim 1, characterized in that the cleaning agent is selected from the group consisting of silicas, aluminum oxides, silicates, metaphosphates and alkaline earth metal carbonates and bicarbonates.

8. The oral and dental hygiene product as claimed in claim 1, characterized in that the product further comprises at least one remineralization promoting component.

9. The oral and dental hygiene product as claimed in claim 8, characterized in that the remineralization promoting component is selected from magnesium salts.

10. The oral and dental hygiene product as claimed in claim 1, characterized in that it comprises as further toothpaste ingredients one or more of surfactants, humectants, binders and active substances that counter dental and gingival disorders.

11. The oral and dental hygiene product as claimed in claim 1, characterized in that the active substances are selected from the group consisting of anticaries active substances, antimicrobial substances, tartar inhibitors, active substances to counter hypersensitive teeth and mixtures of these substances.

12. The oral and dental hygiene product as claimed in claim 1, wherein the calcium salts consist of hydroxyapatite and the protein components consist of Type A gelatin.

13. An oral and dental hygiene product comprising
a) a composite material comprising
hydroxyapatite, in the form of nanoparticulate primary particles having a length of from 5 to 150 nm and a cross section of from 2 to 50 nm,
gelatin, and
b) 0.1 to 9% by weight of a cleaning agent based on the total weight of the product.

14. The oral and dental hygiene product as claimed in claim 13, characterized in that the gelatin is Type A gelatin.

15. The process for cleaning and remineralizing of teeth comprising the step of applying to teeth an oral and dental hygiene product comprising
a) a composite material comprising
calcium salts which are slightly soluble in water, in the form of nanoparticulate primary particles having a length of from 5 to 150 nm and a cross section of from 2 to 50 nm and
protein components selected from the group consisting of proteins, protein hydrolysates and protein hydrolysate derivatives, and
b) 0.1 to 9% by weight of a cleaning agent based on the total weight of product.

16. The process for the cleaning of sensitive teeth comprising the step of applying to the teeth the oral and dental hygiene product as claimed in claim 1.

17. The process for the prevention and control of hypersensitivity in teeth comprising the step of applying to the teeth the oral and dental hygiene product as claimed in claim 1.

18. The process for brightening teeth and preventing discolorations and re-discolorations, comprising the step of applying to the teeth the oral and dental hygiene product as claimed in claim 1.

19. The process for preventing adhesion of plaque to teeth comprising the step of applying to the teeth the oral and dental hygiene product of claim 1.

20. The process for remineralizing of dental lesions in teeth comprising the step of applying to the teeth the oral and dental hygiene product of claim 1.
